SEQUENCE LISTING (3 sequences)

SEQ ID NO: 1 - DNA, 20 bp, Artificial Sequence
  synthetic artificial DNA antigen
gtgtgtgtgtgtgtgtgtgt

SEQ ID NO: 2 - DNA, 20 bp, Artificial Sequence
  synthetic artificial DNA antigen
cacacacacacacacacaca

SEQ ID NO: 3 - protein, 30 aa, Artificial Sequence
  peptide linker
GGEGGGEGEEEGGGEGGEGGEEGGGEEDGG

(19) United States
(12) Patent Application Publication
(10) Pub. No.: US 2019/0008900 A1
(43) Pub. Date: Jan. 10, 2019

(54) METHODS AND REAGENTS TO TREAT AUTOIMMUNE DISEASES AND ALLERGY

(71) Applicant: Tian Xin Wang, Walnut Creek, CA (US)

(72) Inventor: Tian Xin Wang, Walnut Creek, CA (US)

(73) Assignee: Tian Xin Wang, Walnut Creek, CA (US)

(21) Appl. No.: 16/029,594

(22) Filed: Jul. 7, 2018

Related U.S. Application Data
(63) Continuation-in-part of application No. 15/723,173, filed on Oct. 3, 2017.
(60) Provisional application No. 62/529,476, filed on Jul. 7, 2017.

(51) Int. Cl. A61K 35/17; A61K 47/68; C07K 16/28; A61P 37/06

(52) U.S. Cl. CPC A61K 35/17; A61K 47/6849; C07K 16/2887; A61K 2035/122; C07K 16/2827; C07K 16/2818; A61P 37/06; C07K 16/2809

(57) ABSTRACT

Compositions for inducing immune tolerance and methods to modify antigen to treat disease such as autoimmune diseases and allergy are described. The compositions and related methods comprise APC presentable antigens and immunosuppressants that provide tolerogenic immune responses specific to antigen. The compositions can be particle containing antigen and immunosuppressant. The compositions can also be linear polymer containing antigen and immunosuppressant.

### CROSS-REFERENCE TO RELATED APPLICATIONS

This application claims priority to U.S. Provisional Patent Application 62/529,476 filed on Jul. 7, 2017 and is a Continuation-In-Part application of U.S. application Ser. No. 15/723,173 filed on Oct. 3, 2017. The entire disclosure of the prior application is considered to be part of the disclosure of the instant application and is hereby incorporated by reference.

### BACKGROUND OF THE INVENTION

### Field of the Invention

The current invention relates to protein, peptide and antigen modification for pharmaceutical applications and reagents to treat disease such as auto immune disease and allergy. The current invention discloses methods to treat auto immune disease and allergy.

### Background Information

Immune responses are necessary for protection against potentially pathogenic microorganisms. However, undesired immune activation can cause injurious processes leading to damage or destruction of one's own tissues. Undesired immune activation occurs, for example, in autoimmune diseases where antibodies and/or T lymphocytes react with self antigens to the detriment of the body's tissues. This is also the case in allergic reactions characterized by an exaggerated immune response to certain environmental matters and which may result in inflammatory responses leading to tissue destruction. This is also the case in rejection of transplanted organs which is significantly mediated by alloreactive T cells present in the host which recognize donor alloantigens or xenoantigens. Immune tolerance is the acquired lack of specific immune responsiveness to an antigen to which an immune response would normally occur. Typically, to induce tolerance, there must be an exposure to a tolerizing antigen, which results in the death or functional inactivation of certain lymphocytes. This process generally accounts for tolerance to self antigens, or self-tolerance. Immunosuppressive agents are useful in prevention or reduction of undesired immune responses, e.g., in treating patients with autoimmune diseases or with allogeneic transplants. Conventional strategies for generating immunosuppression associated with an undesired immune response are based on broad-acting immunosuppressive drugs. Additionally, in order to maintain immunosuppression, immunosuppressant drug therapy is generally a life-long proposition. Unfortunately, the use of broad-acting immunosuppressants is associated with a risk of severe side effects, such as tumors, infections, nephrotoxicity and metabolic disorders. Accordingly, new immunosuppressant therapies would be beneficial.

### BRIEF DESCRIPTION OF THE DRAWINGS

FIG. 1 shows example of general structure of antigen-drug conjugate

FIG. 2 shows example of general structure of antigen-alpha gal conjugate

FIG. 3 shows an example of antigen-alpha gal conjugate for SLE treatment

FIG. 4 shows examples of 3 different formats of the antigen-drug conjugate.

FIG. 5 shows examples of an antigen-sialic acid rich polymer conjugate to treat autoimmune disease or allergy or to induce immune tolerance.

FIG. 6 shows examples of the conjugate containing antigen and sialic acid/siglec ligand.

FIG. 7 shows schematic example of the structure of the microsphere based agent to induce immune tolerance and treating auto immune diseases or allergy.

FIG. 8 shows different formats of using polymer carrier conjugated with antigen, siglec ligand and other immunosuppressant; and both siglec ligand and other immunosuppressant conjugated to the antigen.

FIG. 9 shows examples of siglec ligand-antigen conjugate for systemic lupus erythematosus treatment.

FIG. 10 shows schematic example of multiple antigens and immunosuppressants with linkers to form a linear polymer.

FIG. 11 shows exemplary scheme of antigen containing polymer conjugated to a nano or micro particle encapsulating immune suppressant.

FIG. 12 shows exemplary scheme of multiple antigens with linkers to form a linear polymer.

FIG. 13 shows exemplary scheme of multiple antigen conjugated to a polymer carrier backbone.

FIG. 14 shows exemplary scheme of antigen containing polymer conjugated to a nano or micro particle.

FIG. 15 shows exemplary scheme of coating additional TB regulatory cell stimulating molecule/cytokine to pMHC-NP/MP.

FIG. 16 shows exemplary scheme of multiple pMHC is conjugated to or expressed in a polymer instead of being coated on a particle.

### DESCRIPTION OF THE INVENTIONS AND THE PREFERRED EMBODIMENT

In one aspect, the current invention discloses a transdermal drug delivery system such as a transdermal patch to treat conditions selected from autoimmune disease, allergy and anti-drug antibody comprising an antigen causing said condition and an immunosuppressant. The antigen can be B cell antigen, T cell antigen in MHC-peptide complex form or the antigen peptide (or its derivative) of T cell antigen that can bind with MHC to form the MHC-peptide complex. Example of immunosuppressant is selected from rapamycin, fujimycin and methotrexate. The current invention also discloses a method to treat autoimmune disease or allergy or inhibit anti-drug antibody production or induce antigen specific immune tolerance in a subject by administering to the subject a said transdermal drug delivery system on the skin.

In another aspect, the current invention discloses a conjugate in linear polymer form or particle form to treat conditions selected from autoimmune disease, allergy and anti-drug antibody or to inhibit anti-drug antibody production or to induce antigen specific immune tolerance comprising an antigen causing the condition, a first immunosuppressant and an optional second immunosuppressant. The antigen can be B cell antigen, T cell antigen in MHC-peptide complex form or the antigen peptide (or its derivative) that can bind with MHC. The first immunosuppressant is selected from siglec ligand such as sialic acid or poly sialic acid. Example of second immunosuppressant is selected from rapamycin, fujimycin, methotrexate and PD-L1. The current invention also discloses a method to treat autoimmune disease or allergy or inhibit anti-drug antibody production or induce antigen specific immune tolerance in a subject by administering to the subject said conjugate (e.g. subcutaneous or intravenous injection).

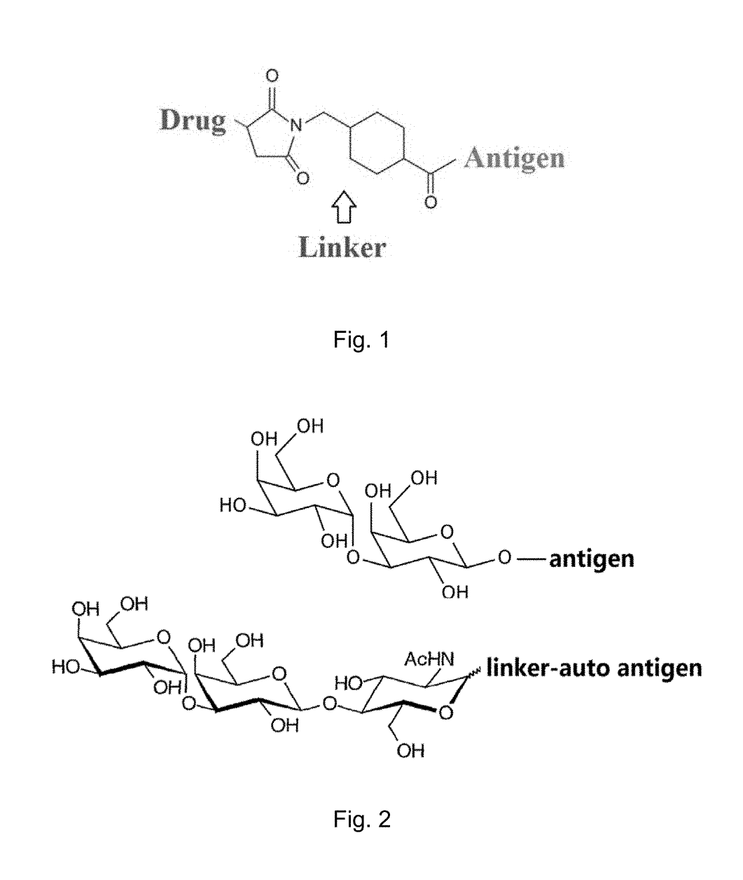

Previous U.S. application Ser. No. 15/723,173 by the inventor discloses antigen-drug conjugate to treat autoimmune diseases and allergy or inhibit anti-drug antibody production or induce antigen specific immune tolerance with general structure as shown in FIG. 1. Auto antibody against DNA is a key pathogenic factor in SLE, DNA coated affinity column is clinically used to remove these Ab from patient blood (hemopurification) as an effective SLE treatment. Antigen-drug conjugate can be used for SLE treatment. DNA-linker-Mertansine (DNA sequence adopted from Abetimus, linker/toxin adopted from Kadcyla, linker can be optimized for B/T cells) is an example of ADC for SLE treatment. The DNA sequence used are the complex formed with GTGTGTGTGTGTGTGTGTGT (SEQ ID NO: 1) and CACACACACACACACACACA (SEQ ID NO: 2). Single strand DNA antigen can also be used to inactivate auto antibody generating cells specific to single strand DNA. It will selectively inactivate the specific B cell clone producing auto antibody against DNA, treat the disease from the source. It can be prepared easily with solid phase synthesis. It can be intravenously injected to the patient having SLE to treat it. Companion test will be performed to increase the efficacy. Patient will be treated with hemopurification to remove the anti-DNA antibody before the first dose ADC administration for better therapeutical index.

Instead of epitope(antigen)-toxin described, epitope (antigen)-alpha gal(e.g. Galactose-alpha-1,3-galactose) can also be used instead, which utilize the endogenous anti gal antibody to inactivate the B cell clone or T cell clone that can selectively bind with the epitope (antigen). The alpha gal can be readily adopted from US patent application Ser. No. 12/450,384 and other publication. Epitope (antigen)-alpha gal conjugate design has the formula: alpha galactosyl-(optional linker)-epitope (antigen), which will allow the T cell/B cell specific to the epitope (antigen) bind with endogenous anti-Gal antibody and therefore be eliminated/inactivated due to the bound antibody. Examples of its structure are shown in FIG. 2.

For example, the antigen can be insulin or insulin fragment that recognized by autoimmune B cell/T cell, or peptide of pancreatic islets recognized by the auto immune T cell in diabetics or the auto antigen of beta cells (e.g. those described in Clin Immunol. 2004 October; 113(1):29-37 and Proc Natl Acad Sci USA. 2003 Jul. 8; 100(14): 8384-8388). This conjugate will selectively inactive the autoimmune B cell/T cells causing diabetics. For T cell antigen, it can be the MHC-peptide complex form, in which the peptide can be optionally covalently linked with the MHC.

The T cell recognize T cell antigen by its TCR receptor. The T cell antigen normally is in the form of MHC-epitope binding complex. The epitope normally is a peptide (sometimes other molecules such as carbohydrate) processed by APC. In some embodiments of the current invention, the antigen for T cells preferably is the formed MHC-epitope complex or its fragment/derivatives/mimics, which has higher specific affinity to TCR than the epitope alone. It can be the monomer form or oligomer (dimer, trimer, tetramer, pentamer or even higher degree oligomer or polymer) form such as the MHC tetramer or dextramer currently used in research. For example, HLA-A2insB10-18 tetramer (described in doi: 10.1073/pnas.0508621102) can be conjugated with the cell inactivating agent with an optional linker to treat Type 1 diabetes by inactivating the auto immune T cell. The epitope (e.g. peptide) can be covalently conjugated with MHC to increase its stability by well known means as disclosed in well known publications. Similarly, the antigen used for B cell in the current invention can also be oligomer or polymer form. However the antigen used for B cell inactivation may not require the MHC component.

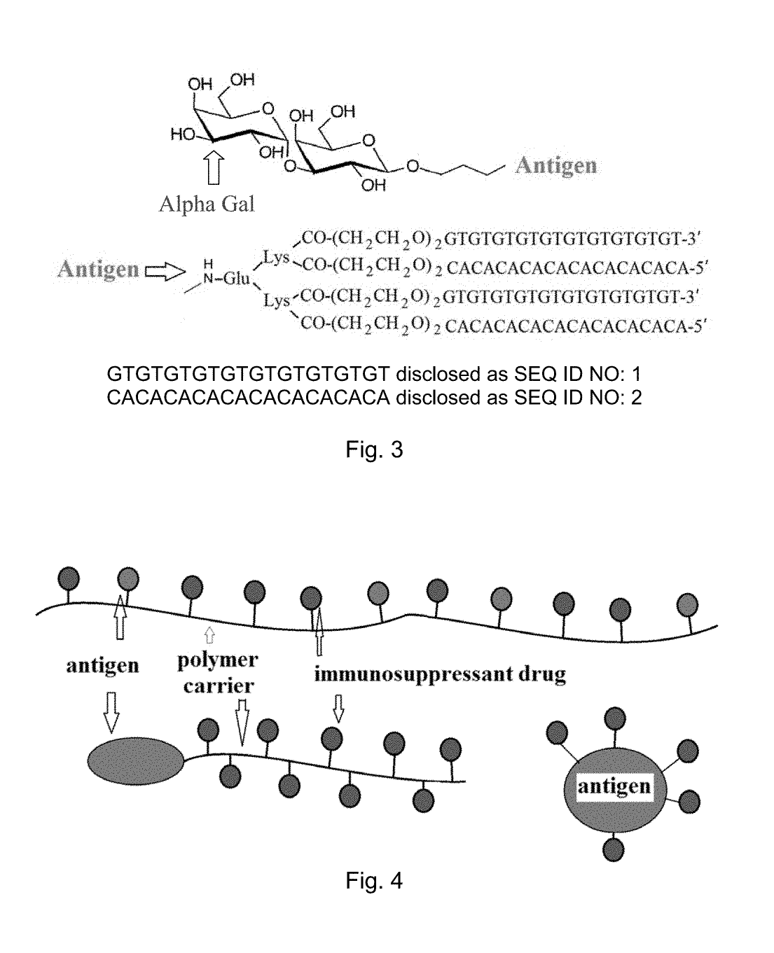

An example reagent that can selectively inactivate B cells producing auto antibody against DNA is shown in FIG. 3, this drug can be used to treat lupus. The patient can receive 500 mg˜1 g of the said conjugate as weekly i.v. injection to treat his lupus until symptom disappears.

A carrier system can be used for the current invention to build the conjugate. For example, the liposome or microparticle or nanoparticle can be used as a carrier. The antigen is immobilized on the surface of the liposome or particles and the effector molecule (e.g. alpha gal, rhamnose, immune suppression cytokine, tregitope peptide, toxin, Si RNA or mi RNA or the like, immune suppressant, antisense molecule) can be either encapsulated inside or co-immobilized on the surface of liposome or particles. The carrier can also be a linear or branched polymer such as dextran. Both antigen and the effector molecule are conjugated to the polymer.

Solid phase particle coated with auto antigen or combinations of different auto antigens for the same diseases (because sometimes a patient will have T cells/B cells specific for a groups of different auto antigens) coated on their surface can be used to treat their corresponding auto immune disease. Because for a specific auto immune diseases sometimes multiple auto antigens are involved (e.g. GAD65, insulin, preproinsulin and etc. for type 1 diabetics), therefore the solid phase particle can be a mixture of different solid phase particle each coated with different auto antigen for this diseases; or a solid phase particle coated with a mixture of the different auto antigen involved for the diseases. However it is known that sometimes a single antigen can be used to induce immune tolerance for a group of devise antigens therefore the mixture of different antigen may not be required. An ELISA test can be performed to the patient to identify the antigens involved and use this information to select suitable solid phase adsorbent for treatment.

When the solid phase particle (e.g. 10 um˜2 mm microparticles or 50 nm˜10 um particles) is coated with MHC-epitope (e.g. peptide) complex (either in monomer or oligomer or polymer form, the complex can be either covalent or non-covalent), it can be used to inactivate T cells against this auto antigen (MHC-epitope complex such as HLA-A2insB10-18).

Instead of alpha gal, other molecule/peptide/protein can also be used to conjugate with a specific antigen to selectively inactivate the specific B cell clone or T cell clone that binds and reacts with the specific antigen. The resulting agent has the general structure:
Cell Inactivating Molecule-Linker (Optional)-Antigen

The agent can be given to the patient (e.g. by i.v. injection) at therapeutic effective amount and in therapeutic acceptable formulation to the patient having autoimmune disease or allergy due to the said antigen to treat said autoimmune disease or allergy or to inhibit anti-drug antibody production or to induce antigen specific immune tolerance. When the antigen is a therapeutic drug (e.g. recombinant protein) or its epitope, it can be given to the patient (e.g. by i.v. injection) to inhibit/prevent the production of anti drug antibody (ADA). It can be used to induce antigen specific immune tolerance. Example of cell inactivating molecule include affinity ligand (e.g. antibody or its fragment, aptamer) or their combination against immune cells (e.g. those used in bi specific antibody and triomab for cancer treatment) such as a antibody against a T-lymphocyte antigen like CD3, or a bi specific antibody (or a triomab having Fc) against CD3 and CD28, or a fusion protein of B7 with an antibody (or its fragment) against CD3, antigen that already has immuno response in the body (e.g. alpha-gal, L-rhamnose), B7, super antigen (e.g. staphylococcal enterotoxin A, SEA), cytokines (e.g. immuno cell inactivating cytokines) and those described in the previous patent applications by the inventor and references. For example, L-rhamnose can be linked with a PEG₃ by a glycoside bond and the PEG₃ is also conjugated with an auto antigen.

When affinity ligand such as antibody or its fragment against cytotoxic immune cell activating receptor such as CD3 of T cell or CD16 of NK cell is conjugated with antigen, it will recruit/activate cytotoxic immune cell such as T cells or NK cells to inhibit/kill the target B/T cell that can bind with the antigen (preferably the antigen for target T cell will be MHC-peptide complex recognized by its TCR); which is similar to the current bi-specific antibody to kill cancer cell except the auto antigen is used in the conjugate instead of the antibody against cancer cell).

For example, in one example an antibody or Fab against CD16A of NK cell (which sequence can be adopted from the TnadAb AFM13 of Affimed) is conjugated with the linker-antigen for SLE shown in FIG. 1 via its cysteine to form a thiol-maleimide linkage, which is widely used in antibody drug conjugate and the conjugation protocol is well known to the skilled in the art. This antigen-anti CD16A antibody conjugate can be used to treat SLE. Once being injected to the patient (e.g. 200 mg˜1000 mg i.v. bi weekly), it will bind with DNA antigen specific B cells and attract NK cell to kill it, therefore inhibit auto antibody production against DNA antigen. Alternatively, an antibody or Fab against CD3 can be used instead of those against CD16 to prepare the conjugate. The resulting conjugate can attract cytotoxic T cell to kill the antigen specific B cell to treat corresponding autoimmune diseases.

Optionally additional affinity ligand can also be introduced into the conjugate to increase the affinity and specificity to B or T cell. For example, antibody against CD20 can also be incorporated in the conjugate via a linker to increase the targeting toward B cell, a scheme similar to tri-specific antibody.

Besides the co-stimulatory molecules B7.1, other co-stimulatory molecules can also be used as cell inactivating molecule such as those selected from other B7 family members including B7.2 (CD86), B7-H1 (PD-L1), B7-H2 (B7RP-1 or ICOS-L or B7h or GL-50), B7-H3 (B7RP-2), B7-H4 (B7x or B7S1), B7-DC (PD-L2) and etc., and these proteins having amino acid sequence of more than 70% identity of the natural and man-made variants. Co-stimulatory molecules B7.1 (CD80) or other co-stimulatory molecule's role is to stimulate the body's immune response. Furthermore, in addition to B7 family members, other molecules can stimulate T cells can also be used as cell inactivating molecule of the present invention. The protocol described in patent application CN102391377A (CN201110338886) can be readily adopted for the current invention. For example, the cytokine of the fusion protein in CN102391377A can be replaced with the auto antigen to generate the conjugate of the current application to inactivate the antigen specific B cell and/or T cells.

B7 is a type of peripheral membrane protein found on activated antigen presenting cells (APC) that, when paired with either a CD28 or CD152 (CTLA-4) surface protein on a T cell, can produce a costimulatory signal or a coinhibitory signal to enhance or decrease the activity of a MHC-TCR signal between the APC and the T cell, respectively. Some type B7 proteins can enhance the activity of T cells (e.g. B7.1, B7.2) and some of them can inhibit the activity of B/T cells (B7.DC/PD-L2, B7.H1/PD-L1). When T cell activating B7 is conjugated with antigen, it will recruit/activate other T cells or cytotoxic immune cells to inhibit/kill (similar to the current bi-specific antibody to kill cancer cell except the auto antigen is used instead of the antibody against cancer cell) the target B/T cell that can bind with the antigen (preferably the antigen for target T cell will be MHC-peptide complex recognized by its TCR). When B/T cell inhibiting B7 is used in the conjugate, it will bind with the corresponding receptors on target B/T cell to kill/inactivate the target B/T cells that can bind with the antigen.

Like B7, other ligand that can activate the inhibitory immune checkpoint receptors on immune cells such as A2AR, B7-H3, B7-H4, BTL, IDO, KIR, LAG3, PD-1, TIM-3 and VISTA, or the ligand (e.g. antibody or its fragment) that can block the activating checkpoint molecules on immune cells such as CD27, CD 47, CD 28, CD40, CD122, CD137, OX40, GITR, CD52 and ICOS, can also be used as cell inactivating molecule. For example the cell inactivating molecule can be PD-L1 or its derivative/fragment or mimic or other ligand that binds to PD-1 to prevent B or T cell activation, PD-L2 or its derivative/fragment or mimic or other ligand that binds to PD-1 to prevent B or T cell activation and etc.

When the target cell is B cell, BCR antigen-TCR antigen conjugate can also be used. Optional linker can be added between these functional groups. In the conjugate the B cell antigen binds with the target B cell and the T cell antigen (MHC-antigen peptide complex, which can be covalently linked together) bind with the effector T cell. The antigen for B cell and T cell can be different. The principle is to recruit the existing effector T cell to kill/inactivate the target B cell. The T cell antigen can also be the peptide that can bind with the MHC to form the MHC-peptide complex or its derivative, instead of the full MHC-peptide complex type T cell antigen, in this case the peptide will be taken by APC and then form the MHC-peptide complex in vivo

The current invention further discloses methods and regents to treat autoimmune diseases and allergy or to inhibit anti-drug antibody production or to induce antigen specific immune tolerance by applying the combination of antigen and immunosuppressive agent/drug either as a physical mixture or as synthetic conjugate or as nano/micro particles or liposome to the object/patient in need. The term nano/micro particle means the particle is in either nanometer or micrometer range of size (diameter). For example, the nano/micro particle can be in the size range of 50 nm˜100 um. List of exemplary immunosuppressive drugs can be found at “Immunosuppressive drug” article page in Wikipedia. The immunosuppressive agent/drug (immunosuppressants) suitable for the current application include but are not limited to, statins; mTOR inhibitors, such as rapamycin or a rapamycin analog; TGF-β signaling agents; TGF-β receptor agonists; TLR (toll like receptor) inhibitors; Pattern recognition receptor inhibitors; NOD-like receptors (NLR) inhibitors; RIG-I-like receptors inhibitors; NOD2 inhibitors; histone deacetylase inhibitors, such as Trichostatin A; corticosteroids; inhibitors of mitochondrial function, such as rotenone; P38 inhibitors; NF-κβ inhibitors, such as 6Bio, Dexamethasone, TCPA-1, IKK VII; adenosine receptor agonists; prostaglandin E2 agonists (PGE2), such as Misoprostol; phosphodiesterase inhibitors, such as phosphodiesterase 4 inhibitor (PDE4), such as Rolipram; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3 KB inhibitors, such as TGX-221; autophagy inhibitors, such as 3-Methyladenine; aryl hydrocarbon receptor inhibitors; proteasome inhibitor I (PSI); and oxidized ATPs, such as P2X receptor blockers. Immunosuppressants also include IDO, vitamin D3, cyclosporins, such as cyclosporine A, aryl hydrocarbon receptor inhibitors, resveratrol, azathiopurine (Aza), 6-mercaptopurine (6-MP), 6-thioguanine (6-TG), FK506, sanglifehrin A, salmeterol, mycophenolate mofetil (MMF), aspirin and other COX inhibitors, niflumic acid, estriol and triptolide, siglec ligand such as sialic acid and its derivative including poly sialic acid sialic acid-lipid conjugate. In embodiments, the immunosuppressant may comprise any of the agents provided herein. The immunosuppressant can be a compound that directly provides the immunosuppressive (e.g., tolerogenic) effect on APCs or it can be a compound that provides the immunosuppressive (e.g., tolerogenic) effect indirectly (i.e., after being processed in some way after administration). Immunosuppressants, therefore, include prodrug forms of any of the compounds provided herein.

The immunosuppressant also include Heme Oxygenase-1 (HO-1) inducer such as Cobalt protoporphyrin (CoPP), protoporphyrin IX containing a ferric iron ion (Heme B) with a chloride ligand (Hemin), hematin, iron protoporphyrin or heme degradation products as well as those described in PCT/EP2015/074819. Siglecs (Sialic acid-binding immunoglobulin-type lectins) ligand such as sialic acid or its derivatives is also another type of immunosuppressant that can be used in current invention. PD-L1 is also another type of immunosuppressant that can be used in current invention. PD-L1 can effectively inhibit cytotoxic T cell. Fragment or mimic or derivative of PD-L1 that can bind with PD-1 can also be used instead. Other inhibitory ligands that can bind with inhibitory checkpoint receptor (e.g. A2AR, BTLA, CTLA-4, CD 47, KIR, LAG3, TIM-3, VISTA and etc) such as B7-H3, B7-H4 can also be used instead of PD-L1. Molecule that can promote T/B reg expansion (e.g. cytokine that can stimulate T/B reg expansion such as IL-2 and TGF-β) is also another type of immunosuppressant. Different immunosuppressant can be used as a mixture and be used in combination in the current invention.

The immunosuppressant can be a compound that directly provides the immunosuppressive (e.g., tolerogenic) effect on APCs or it can be a compound that provides the immunosuppressive (e.g., tolerogenic) effect indirectly (i.e., after being processed in some way after administration). Immunosuppressants, therefore, include prodrug forms of any of the compounds provided herein.

Immunosuppressants also include nucleic acids that encode the peptides, polypeptides or proteins provided herein that result in an immunosuppressive (e.g. tolerogenic) immune response. In embodiments, therefore, the immunosuppressant is a nucleic acid that encodes a peptide, polypeptide or protein that results in an immunosuppressive (e.g., tolerogenic) immune response. The nucleic acid can be coupled to synthetic nanocarrier. The nucleic acid may be DNA or RNA, such as mRNA. In embodiments, the inventive compositions comprise a complement, such as a full-length complement, or a degenerate (due to degeneracy of the genetic code) of any of the nucleic acids provided herein. In embodiments, the nucleic acid is an expression vector that can be transcribed when transfected into a cell line. In embodiments, the expression vector may comprise a plasmid, retrovirus, or an adenovirus amongst others. Nucleic acids can be isolated or synthesized using standard molecular biology approaches, for example by using a polymerase chain reaction to produce a nucleic acid fragment, which is then purified and cloned into an expression vector.

In some embodiments, the immunosuppressants provided herein are coupled to synthetic nanocarriers or microcarriers. In preferable embodiments, the immunosuppressant is an element that is in addition to the material that makes up the structure of the synthetic nanocarrier or microcarrier. For example, in one embodiment, where the synthetic nanocarrier or microcarrier is made up of one or more polymers, the immunosuppressant is a compound that is in addition and coupled to the one or more polymers. As another example, in one embodiment, where the synthetic nanocarrier or microcarrier is made up of one or more lipids, the immunosuppressant is again in addition and coupled to the one or more lipids. In embodiments, such as where the material of the synthetic nanocarrier or microcarrier also results in an immunosuppressive (e.g., tolerogenic) effect, the immunosuppressant is an element present in addition to the material of the synthetic nanocarrier or microcarrier that results in an immunosuppressive (e.g., tolerogenic) effect.

Other exemplary immunosuppressants include, but are not limited, small molecule drugs, natural products, antibodies (e.g., antibodies against CD20, CD3, CD4), biologics-based drugs, carbohydrate-based drugs, nanoparticles, liposomes, RNAi, antisense nucleic acids, aptamers, methotrexate, NSAIDs; fingolimod; natalizumab; alemtuzumab; anti-CD16, anti-CD3; tacrolimus (FK506), etc. Further immunosuppressants, are known to those of skill in the art, and the invention is not limited in this respect. Additional immunosuppressants can be found in Patent and patent application U.S. Ser. No. 13/880,778, U.S. Ser. No. 14/934,135, CA 2910579, U.S. Ser. No. 13/084,662, U.S. Ser. No. 14/269,048, U.S. Pat. No. 8,652,487 and other patent application filed by Selecta Biosciences.

Additional immunosuppressants can be found in Patent WO2012054920A2, Patent WO2016073799A1, WO2012149393 A3, Patent WO2014179771A1, PCT/US2012/035405, Patent US20110262491, U.S. Pat. No. 8,652,487 and other patent application filed by Selecta Biosciences. Selecta's publications disclose synthetic nanocarrier methods, and related compositions, comprising B cell and/or MHC Class II-restricted epitopes and immunosuppressants in order to generate tolerogenic immune responses. In their disclosure, the antigen/epitope is conjugated to the nanocarrier and immunosuppressants is coupled to the nanocarrier.

An alternative method and composition is to use nano/micro particle having antigen/epitope non-covalently adsorbed to its surface and immunosuppressant encapsulated within. The nano/micro particles can be made of biodegradable materials such as PLGA. These kinds of nano/micro particles (e.g. 10 nm 10 um of diameter in size) can be given to the patient in need as injection or inhaler or applied topically to induce immuno tolerance. The encapsulation of immunosuppressant is well known to the skilled in the art and can be adopted from related publications readily. The surface of the nano/micro particles can have charged groups such as amino or carboxyl group to increase the binding of antigen/epitope to its surface; it can also have a hydrophobic surface to allow binding antigen/epitope via hydrophobic interaction; or the combination of them. Introducing charged groups to the surface can be done by using surface modification or using amine or carboxyl group containing molecules to prepared the nano/micro particles. The antigen/epitope can also be conjugated with a lipid molecule such as fatty acid or cholesterol to increase its binding to nano/micro particles. The adsorption of antigen/epitope to the nano/micro particle surface can be done by incubating antigen/epitope with the nano/micro particle (e.g. 4 degree overnight in aqueous solution buffer such as 1×PBS) and then removing the unbound antigen/epitope (e.g. washing the nano/micro particle with aqueous buffer several times, similar to the ELISA plate coating procedure). In one example, 50 nm˜200 nm size PLGA nano particle encapsulated with 10% by weight of rapamycin is prepared according to the literature. Next the PLGA nano particle is mixed with OVA (10 mg/mL) at 4 C overnight to generate the OVA (ovalbumin) coated particle. The particle is washed 3 times with PBS to remove unbound OVA. In another example, rapamycin is dissolved in DMSO at 50 mg/ml. A total of 50 μL rapamycin is added to 1 ml PLGA (5 mg/ml) dissolved in dichloromethane. Next the mixture is homogenized with 0.4 ml 5% OVA solution for 10 min using ultrasonication. The o/w emulsion is added to 2.1 ml of a 5% w/v solution of PVA to evaporate the organic solvent for 4 h at room temperature. OVA coated nano particles containing rapamycin are obtained after centrifugation at 3,500 g for 20 min. Additional washing step can be performed to obtain unbound OVA free particles. This OVA coated particle can be given to the target in need to induce OVA immune tolerance, using the similar protocol described in the publications (e.g. those from Selecta Bio). The OVA can be replaced with other antigen/epitope molecule to induce corresponding immune tolerance. In another sample, lipophilic carboxylic acid or lipophilic amine or anionic detergent or cationic detergent (e.g. fatty acid such as caprylic acid, lauric acid; or cationic lipid such as DOTMA, DOTAP, cholesterylamine) can be added to the PLGA to prepare PLGA particle having surface charge. In one example, rapamycin is dissolved in DMSO at 50 mg/ml with lauric acid at 10 mg/mL. A total of 50 μL rapamycin/lauric acid is added to 1 ml PLGA (5 mg/ml PLGA) dissolved in dichloromethane. Next the mixture is homogenized with 0.1 ml 2% caprylic acid solution for 10 min using ultrasonication. The o/w emulsion is evaporated to remove the organic solvent for 4 h at room temperature. The resulting PLGA particle is washed 3 times with PBS and then incubated with OVA to prepare OVA bound particles.

Furthermore, antigen/epitope can also be encapsulated within the nano/micro particle besides being conjugated or adsorbed to its surface. The preparation of antigen/epitope encapsulation is well known to the skilled in the art and can be adopted from related publications readily, e.g. using a double emulsion water/oil/water system.

US patent application 20130287729 A1 disclosed antigen-specific, tolerance-inducing microparticles and uses thereof. It disclosed a microparticle (0.5 μm-10.0 μm in size) for targeting an antigen-presenting immune cell of interest and for inducing antigen-specific immune tolerance, wherein the microparticle comprises an antigen and a therapeutic agent wherein the therapeutic agent is an immunomodulatory agent, an immunosuppressive tolerogenic agent, or an agent that recruits the antigen-presenting immune cell of interest, wherein the surface of the microparticle comprises a ligand that targets the antigen-presenting immune cell of interest and the microparticle is made of biodegradable material. A further improvement of this method and composition is to use a nano/micro particle having the size of 50 nm˜5 um, preferably made of biodegradable materials. In some embodiments, the surface of the nano/micro particle is coated with Fc portion of an antibody or a full antibody with its Fc portion facing outside. This will bind with the FcR to facilitate APC uptake. In other embodiments, the surface of the nano/micro particle needs not to have a ligand that targets the antigen-presenting immune cell. In some embodiments, it can have antigen/epitope coated on its surface. The inner part of the nano/micro particle contains immunosuppressive agent listed in the current application and optionally antigen/epitope, e.g. by encapsulation. The preparation method is well known to the skilled in the art and can be adopted from related publications readily.

US patent application 20160338953 A1 disclosed a liposome-based immunotherapy. It provided a liposome encapsulating an autoantigen, wherein the liposome has a size comprised from 500 to 15000 nm and the liposome membrane comprises phosphatydilserine (PS) in an amount comprised from 10 to 40% by weight with respect to the total membrane liposomal composition. Pharmaceutical or veterinary compositions comprising a therapeutically effective amount of said liposome were also provided. Further, it provided liposomes and pharmaceutical or veterinary compositions as defined above for use as a medicament, particularly for the treatment of autoimmune diseases. Finally it provided liposomes and pharmaceutical or veterinary compositions as defined above for use in the restoration of tolerance to self in a patient suffering from an autoimmune disease.

The current invention also discloses antigen-specific, tolerance-inducing liposome and uses thereof. The liposome contains immunosuppressive agent listed in the current application (and optionally antigen/epitope molecule) inside by encapsulation. Optionally the surface of the liposome can also have antigen/epitope coated. It can be given to the patient in need as injection or inhaler or applied topically to induce immuno tolerance. The lipid used for liposome can include but not limited to phosphatydilserine at 10 to 40% by weight of the membrane. It can also use non-phosphatydilserine lipid to prepare the membrane. The antigen/epitope can also be conjugated with a lipid type molecule such as fatty acid or phospholipid or cholesterol derivative to allow it to be inserted to the liposome membrane. Suitable liposome can have a size between 50 nm˜20 um. The preparation method and the protocol of its use are well known to the skilled in the art and can be adopted from related publications readily such as those in US20160338953. Example of the lipid molecule suitable for the current invention to prepare liposome includes but is not limited to phospholipid glycerolipid, glycerophospholipid, sphingolipid, ceramide, glycerophosphoethanolamine, sterol or steroid. These lipid molecules can also be used to prepare the antigen/epitope-lipid conjugate. Membrane anchoring peptide-antigen/epitope conjugate can also be used instead of antigen/epitope-lipid conjugate.

In addition, other molecule that can promote TB reg expansion (e.g. IL-2 and/or TGF-β and PD-L1) can also be coated/conjugated to and/or encapsulated within the liposome and nano/micro particle.

The current invention discloses methods and regents to treat autoimmune diseases and allergy by applying the mixture of antigen and immunosuppressive agent topically to the object/patient in need. It can also be used to inhibit the generation of anti drug antibody when the antigen is the drug (e.g. a protein drug) or its epitope. It will induce immune tolerance for the antigen. Examples of the formulation suitable for the current application include solid form such as powder, gel, lotion, ointment, solution, spray, suppository, lozenge, tablet and patch that can be topically applied to the skin or mucosa. The term topical drug delivery include drug delivery route other than injection. It includes applying drug to skin or mucosa. It includes intranasal delivery, rectal delivery, sublingual delivery and oral mucosa delivery. The immunosuppressive agent can be in the form of active agent, prodrug form, micro particle or nano particle form or liposome form. The antigen can be either B cell antigen/epitope or T cell antigen/epitope (e.g. MHC-peptide complex or conjugate; or the peptide antigen that can bind with MHC) or their combination. The combination can be either B cell antigen/epitope with T cell antigen/epitope; or the combination of several different B cell antigen/epitope and/or several different T cell antigen/epitope targeting the same disease or different diseases. The use of peptide antigen (T cell epitope) that can bind with MHC to form MHC-peptide complex in vivo (T cell antigen) instead of the peptide-MHC complex reduce the size and molecular weight, therefore improve the transdermal delivery. Examples of them can be found in the current application and related publications and patent applications.

In some embodiments, the method is to use a patch containing both antigen/allergen and immune suppressive drug (the drug listed above such as rapamycin or fujimycin or methotrexate or sialic acid or its derivative or high affinity siglec binder or their combination). The sialic acid can be either free sialic acid or sialic acid ester, sialic acid-lipid conjugate from. For example, sialic acid can be conjugated to cholesterol to form an ester bond using the —COOH of sialic acid with the —OH of the cholesterol. This conjugate will have better trandermal and cell membrane permeation capability. The fatty acid can also be conjugated with sialic acid's —OH to form the conjugate. These conjugate will work as immune suppressive drug after being transdermally delivered. Examples of high affinity Siglec ligands can be found in U.S. Pat. No. 8,357,671.

The transdermal or transmucosal delivery of both antigen and immunosuppressive drug will induce immune tolerance via DC cells in the skin or mucosa. The skin may be exfoliated to remove stratum corneum layer to increase drug delivery or using a micro needle system. This would be a much easier strategy for food allergy and auto immune diseases treatment than injection. The skin may be intact or may be exfoliated to remove stratum corneum layer to increase drug delivery. Micro needle system can also be used to the skin. The micro needle in the micro needle system can be made of bio degradable material such as PLGA encapsulating antigen and immunosuppressant. Alternatively, a bio degradable implant encapsulating antigen and immunosuppressant can also be used. The size of the implant can be bigger than 10 um in diameter, preferably >100 um, if the implant is a macro particle. For example, a 2 mm (length)×0.3 mm (diameter) rod made with PLGA containing 3 mg rapamycin and 1 mg gliadin can be used as an implant underneath the skin to treat gluten intolerance. Other implant format can also be used such as NanoPortal Capsule from Nanoprecision Medical and Medici Drug Delivery System from Intarcia, as long as they can deliver the antigen and immunosuppressant simultaneously.

DBV Technologies and other groups (e.g. those described in Epicutaneous Immunotherapy for Aeroallergen and Food Allergy DOI: 10.1007/s40521-013-0003-8) are using skin patch containing allergen to treat allergy by inducing tolerance for the antigen (allergen). The topically patch or other formulation can be readily adopted for the current application. For example, the topical applied formulation such as patch described in U.S. Ser. No. 15/135,914, U.S. Pat. No. 6,676,961, U.S. Ser. No. 15/111,204, U.S. Pat. No. 8,932,596B2, U.S. Ser. No. 15/184,933A1 and U.S. Pat. No. 8,202,533B2 can be adopted for the current application by adding additional immune suppressive drug in the patch (e.g. 0.1 mg-20 mg of rapamycin or fujimycin or 1 mg-100 mg methotrexate or their directives or prodrug) as well as those commercial available patch (e.g. VIASKIN® MILK and VIASKIN® PEANUT). The administration method can be essentially the same as the prior arts except the patch contains immunosuppressants. Additional transdermal enhancer (e.g. DMSO, Azone, fatty acid, hyaluronic acid and etc, which can be found in the publication readily as well as their suitable amount) can be added to the patch or applied to the skin before applying the patch. Example of transdermal enhancing agent can be added include DMSO (e.g. 10˜300 mg/patch), azone (e.g. 1%˜10% of total drug weight), surfactant, fatty acid (e.g. 1%˜10% oleic acid). The skin can also remove for stratum corneum with be exfoliation or other means to enhance the transdermal delivery. In one example, the patch contains 500 ug-10 mg gluten (e.g. G5004 Gluten from wheat, Sigma) and 0.1 mg˜10 mg of rapamycin or 1 mg-50 mg methotrexate. For example, antigen such as gluten and immunosuppressant such as rapamycin and/or methotrexate can be in powder form, which can be simply mixed together physically, they can also be co-dissolved and then dried and then placed in the patch. In another example, the patch contains 5 mg gluten (e.g. G5004 Gluten from wheat, Sigma) and 5 mg of rapamycin or 50 mg methotrexate and optionally additional 30 mg azone. In another example, the patch contains 5 mg gluten (e.g. G5004 Gluten from wheat, Sigma) and 100 mg of sialic acid or sialic acid-cholesterol conjugate or 10 mg methotrexate. This can be used to induce gluten tolerance and treat gluten intolerance. The gluten can be replaced with gliadin instead. In embodiments, the patch can be applied daily for 1-5 weeks. In another example, the antigen is peanut antigen ara h2 200 ug and 2 mg of rapamycin is in the patch to treat peanut allergy. In one example, peanut antigen ara h2 200 ug, 2 mg of rapamycin and 50 mg sucrose is dissolved in water and then lyophilized and then placed in the patch. In one example, peanut antigen ara h2 200 ug, 2 mg of rapamycin, 50 mg SDS and 50 mg sucrose is dissolved in water and then lyophilized and then placed in the patch. In one example, peanut antigen ara h2 200 ug, 2 mg of rapamycin, 100 mg DMSO and 50 mg sucrose is dissolved in water and then lyophilized and then placed in the patch. In another example, the antigen is the double strand DNA (1 mg˜10 mg) in the previous figures to treat lupus and the drug is 3 mg of rapamycin or fujimycin or Temsirolimus. In another example, the nasal spray contains 1 mg gluten (e.g. G5004 from Sigma, Gluten from wheat) and 1 mg of rapamycin or 10 mg methotrexate in a suitable form for each spray. In another example, the sublingual lozenge contains 50 mg gluten (e.g. G5004 from Sigma, Gluten from wheat) and 1 mg of rapamycin or 20 mg methotrexate. In another example, the gel contains 50 mg gluten (e.g. G5004 Gluten from wheat, Sigma) and 2 mg of rapamycin or 20 mg methotrexate in each 1 ml of gel. The immunosuppressant drug or both the immunosuppressant drug and the antigen can be either in the form of powder or gel or semi liquid or in the form of liposome (e.g. 100 nm˜5 um diameter) or in a nano/micro particle (e.g. 100 nm˜1 um) or being conjugated to a dendrimer or linear polymer (e.g. couple to poly acrylic acid or poly Sialic acid via ester bond to form a polymer based prodrug with MW=5K˜500K).

Other pharmaceutically acceptable amount of antigen and immunosuppressant can also be used in the patch, as long as it can produce satisfactory biological and therapeutical (e.g. immune tolerance) effect, which can be determined experimentally by screening and testing with well-known protocol and methods.

The antigen can be either B cell antigen/epitope or T cell antigen/epitope (e.g. MHC-peptide complex or conjugate; or the peptide antigen that can bind with MHC) or their combination. Examples of them can be found in the current application and related publications and patent applications.

The transdermal delivery of both antigen and immunosuppressive drug will be uptaken by APC in the skin, induce/activate tolerogenic dendritic cell and Treg/Breg, inhibit B cell activation/antibody production, germinal centre formation and antigen-specific hypersensitivity reactions, resulting in long term antigen specific immune tolerance.

A skin patch (also called transdermal patch) is a medicated adhesive patch or attachable patch that is placed on the skin to deliver a specific dose of medication through the skin and into the bloodstream. A wide variety of pharmaceuticals are now available in transdermal patch form.

There are several main types of skin/transdermal patches. The Single-layer Drug-in-Adhesive type is that the adhesive layer of this system also contains the drug. In this type of patch the adhesive layer not only serves to adhere the various layers together, along with the entire system to the skin, but is also responsible for the releasing of the drug. The adhesive layer is surrounded by a temporary liner and a backing. The Multi-layer Drug-in-Adhesive type is the multi-layer drug-in-adhesive patch is similar to the single-layer system; the multi-layer system is different, however, in that it adds another layer of drug-in-adhesive, usually separated by a membrane (but not in all cases). One of the layers is for immediate release of the drug and other layer is for control release of drug from the reservoir. This patch also has a temporary liner-layer and a permanent backing. The drug release from this depends on membrane permeability and diffusion of drug molecules. The Reservoir type is unlike the single-layer and multi-layer drug-in-adhesive systems, the reservoir transdermal system has a separate drug layer. The drug layer can be a liquid or gel or powder compartment containing a drug solution or suspension or powder separated by the adhesive layer. This patch is also backed by the backing layer. In this type of system the rate of release is zero order. The Matrix type has a drug layer of a solid or semisolid matrix containing a drug solution or suspension or solid layer such as powder or film. The adhesive layer in this patch surrounds the drug layer, partially overlaying it. In some embodiments, the reservoir type and the matrix type can be used for current invention.

In one example, antigen and immunosuppressant loaded matrix-type transdermal patch is prepared by using solvent casting method. A petri dish with a total area of 50 cm2 is used. Antigen and immunosuppressant are dissolved in 5 mL of water, methanol (1:1) solution and mixed until clear solution is obtained. 200 mg polyethylene glycol 400 is used as plasticizer and optional 100 mg propylene glycol or oleic acid or tween 80 is used as permeation enhancer, together with 100 mg sucrose they are added to the antigen/immunosuppressant solution. The resulted uniform solution is cast on the petri dish, which is lubricated with glycerin and lyophilized or dried at room temperature for 24 h. Next the dried patch is placed on a cellulose acetate membrane used as backing membrane. In another example, weighed amount of PVA (2.5% w/v) is added to a distilled water and a homogenous solution is made by constant stirring and intermittent heating at 60° C. for a few seconds and poured into glass molds already wrapped with aluminium foil around open ends and are kept for drying at 60° C. for 6 h, forming a smooth, uniform, and transparent backing membrane. Backing membrane is used as a support for antigen and immunosuppressant containing matrix.

In some embodiments, the skin patch device used in the method of the invention preferably comprises a backing, the periphery of said backing being adapted to create with the skin a hermetically closed chamber. This backing bears on its skin facing side within the chamber the composition used to decrease the skin reactivity. Preferably, the periphery of the backing has adhesive properties and forms an airtight joint to create with the skin a hermetically closed chamber.

In a particular embodiment, the composition allergens and immunosuppressants are maintained on the backing by means of electrostatic and/or Van der Waals forces. This embodiment is particularly suited where the composition allergens are in solid or dry form (e.g., particles), although it may also be used, indirectly, where the allergens are in a liquid form. Within the context of the present invention, the term “electrostatic force” generally designates any non-covalent force involving electric charges. The term Van der Waals forces designates non-covalent forces created between the surface of the backing and the solid allergen, and may be of three kinds: permanent dipoles forces, induced dipoles forces, and London-Van der Waals forces. Electrostatic forces and Van der Waals forces may act separately or together. In this respect, in a preferred embodiment, the patch device comprises an electrostatic backing. As used herein, the expression “electrostatic backing” denotes any backing made of a material capable of accumulating electrostatic charges and/or generating Van der Waals forces, for example, by rubbing, heating or ionization, and of conserving such charges. The electrostatic backing typically includes a surface with space charges, which may be dispersed uniformly or not. The charges that appear on one side or the other of the surface of the backing may be positive or negative, depending on the material constituting said backing, and on the method used to create the charges. In all cases, the positive or negative charges distributed over the surface of the backing cause forces of attraction on conducting or non-conducting materials, thereby allowing to maintain the allergen and immunosuppressant. The particles also may be ionized, thereby causing the same type of electrostatic forces of attraction between the particles and the backing. Examples of materials suitable to provide electrostatic backings are glass or a polymer chosen from the group comprising cellulose plastics (CA, CP), polyethylene (PE), polyethylen terephtalate (PET), polyvinyl chlorides (PVCs), polypropylenes, polystyrenes, polycarbonates, polyacrylics, in particular poly(methyl methacrylate) (PMMA) and fluoropolymers (PTFE for example). The foregoing list is in no way limiting.

The back of the backing may be covered with a label which may be peeled off just before application. This label makes it possible, for instance, to store the composition allergen in the dark when the backing is at least partially translucent. The intensity of the force between a surface and a particle can be enhanced or lowered by the presence of a thin water film due to the presence of moisture. Generally, the patch is made and kept in a dry place. The moisture shall be low enough to allow the active ingredient to be conserved. The moisture rate can be regulated in order to get the maximum adhesion forces. As discussed above, the use of an electrostatic backing is particularly advantageous where the allergen is in a dry form, e.g., in the form of particles. Furthermore, the particle size may be adjusted by the skilled person to improve the efficiency of electrostatic and/or Van der Waals forces, to maintain particles on the support.

In a specific embodiment, the patch comprises a polymeric or metal or metal coated polymeric backing and the particles of composition allergens are maintained on the backing essentially by means of Van der Waals forces. Preferably, to maintain particles on the support by Van der Waals forces, the average size of the particles is lower than 60 micrometer. In another embodiment, the allergens are maintained on the backing by means of an adhesive coating on the backing. The backing can be completely covered with adhesive material or only in part. Different occlusive backings can be used such as polyethylene or PET films coated with aluminium, or PE, PVC, or PET foams with an adhesive layer (acrylic, silicone, etc.). Examples of patch devices for use in the present invention are disclosed in patent application U.S. Ser. No. 11/915,926 or U.S. Pat. No. 7,635,488.

Other examples are disclosed in patent application U.S. Ser. No. 13/230,689, which also discloses a spray-drying process to load the substance in particulate form on the backing of a patch device. An electrospray device uses high voltage to disperse a liquid in the fine aerosol. Allergens and immunosuppressants dissolved in a solvent are then pulverized on the patch backing where the solvent evaporates, leaving allergens and immunosuppressants in particles form. The solvent may be, for instance, water or ethanol, according to the desired evaporation time. Other solvents may be chosen by the skilled person. This type of process to apply substances on patch backing allows nano-sized and mono-sized particles with a regular and uniform repartition of particles on the backing. This technique is adapted to any type of patch such as patch with backing comprising insulating polymer, doped polymer or polymer recovered with conductive layer. Preferably, the backing comprises a conductive material.

In another embodiment, the periphery of the backing is covered with a dry hydrophilic polymer, capable of forming an adhesive hydrogel film by contact with the moistured skin (as described in U.S. Ser. No. 12/680,893). In this embodiment, the skin has to be moistured before the application of the patch. When the hydrogel comes into contact with the moistured skin, the polymer particles absorb the liquid and become adhesive, thereby creating a hermetically closed chamber when the patch is applied on the skin. Examples of such hydrogels include polyvinylpyrolidone, polyacrylate of Na, copolymer ether methyl vinyl and maleic anhydride.

In another particular embodiment, the liquid composition allergen and immunosuppressant is held on the support of the patch in a reservoir of absorbent material. The composition may consist in an allergen+immunosuppressant solution or in a dispersion of the mixture, for example in glycerine. The adsorbent material can be made, for example, of cellulose acetate. The backing may be rigid or flexible, may or may not be hydrophilic, and may or may not be translucent, depending on the constituent material. In the case of glass, the support may be made break-resistant by bonding a sheet of plastic to the glass. In one embodiment, the backing of the patch contains a transparent zone allowing directly observing and controlling the inflammatory reaction, without necessarily having to remove the patch. Suitable transparent materials include polyethylene film, polyester (polyethylene-terephtalate) film, polycarbonate and every transparent or translucent biocompatible film or material.

Current invention also discloses methods and regents to treat autoimmune diseases and allergy or to inhibit anti-drug antibody production or to induce antigen specific immune tolerance by applying the mixture of said antigen and said immunosuppressive agent/drug as injection to the object/patient in need. The injection can be given as either subcutaneous injection or intramuscular injections or intradermal injections. The injection contains a viscosity enhancing agent to increase its viscosity after being injected, which acts as a sustained release formulation of both antigen and immunosuppressive agent. Molecule that can promote TB reg expansion (e.g. IL-2 and/or TGF-β and.or PD-L1) can also be added into the injection in combination with other immunosuppressive agent. Antigen and immunosuppressive agent can be either in free molecule form or in nano/micro particle from including liposome form. In certain embodiments, the injection has a viscosity greater than 10,000 cps at room temperature. In certain embodiments, the injection has a viscosity greater than 100,000 cps at room temperature. In certain embodiments, the injection has a viscosity greater than 5,000,000 cps at room temperature. In certain embodiments, the injection has a viscosity of 11,000,000 cps at room temperature. Example of the viscosity enhancing agent can be found readily from known pharmaceutical acceptable excipient such as hyaluronic acid, starch and carbomer. In some embodiments, the viscosity enhancing agent is biodegradable. In one example, a viscous injection contains 5 mg/mL gluten (e.g. G5004 Gluten from wheat, Sigma) and 5 mg/mL of rapamycin or 50 mg/mL methotrexate and suitable amount of hyaluronic acid (e.g. 50 mg/mL) to reach a viscosity of 5,000,000 cps with optional 1 mg/mL IL-2. The injection formulation can also be a thermal phase changing formulation. Thermal phase changing formulation is a formulation that change its phase from liquid at low temperature or room temperature (25 C) to semisolid/gel when temperature increases to body temperature (37 C), which can use poloxamer as excipient. A thermal phase changing injectable formulation containing both antigen and immunosuppressive agent can be given as either subcutaneous injection or intramuscular injections or intradermal injections to induce antigen specific immune tolerance and treat corresponding auto immune diseases or allergy. It has low viscosity at low or room temperature but high viscosity at body temperature. The preparation of this kind of thermal phase changing injectable formulation can be adopted from related publications readily by the skilled in the art.

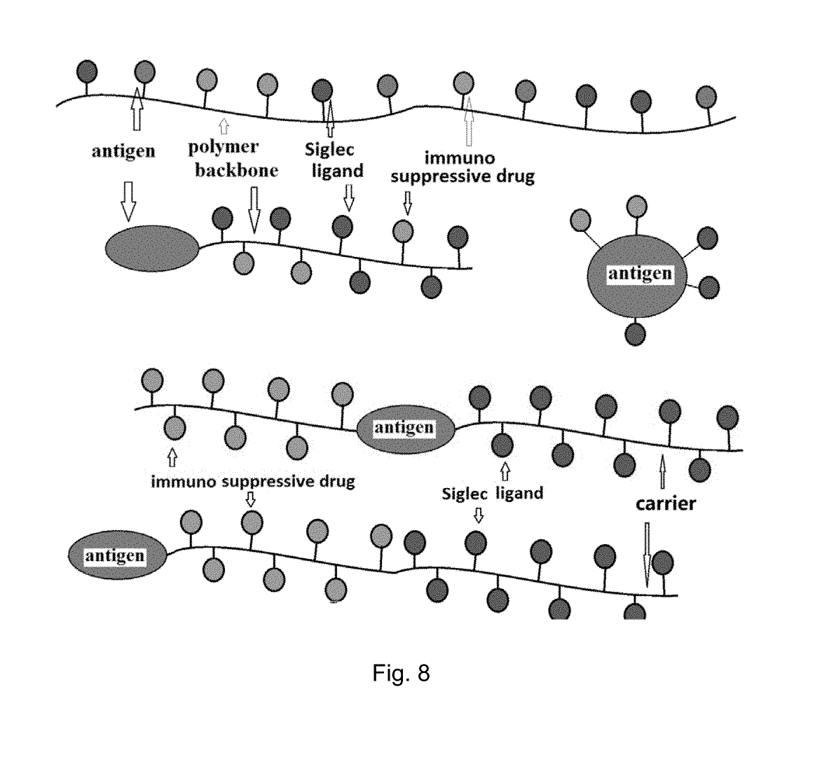

The immunosuppressive agent can also be conjugated to carbohydrate polymer to form prodrug as described in U.S. application Ser. No. 15/723,173. The novel prodrugs can be in the form of carbohydrate (or other polymer) drug conjugate in which the drug is conjugated to the carbohydrate (or other polymer) with cleavable linkage. More than one drugs can be conjugated to the polymer backbone. Suitable carbohydrate includes sialic acid containing polymer, hyaluronic acid, chondroitin sulfate, dextran, carboxyl dextran, cellulose, carboxyl cellulose and their derivatives. In some embodiments, preferably the carbohydrate is selected from sialic acid containing polymer, hyaluronic acid, starch, dextran and chondroitin sulfite. The sialic acid containing polymer suitable for the current invention include poly sialic acid formed by sialic acid monomer connected with α2,3 or α2,6 or α2,8 or α2,9 linkage or their combination. It also includes graft polymer or branched polymer containing sialic acid. It can also be a linear polymer backbone (e.g. dextran or synthetic polymer such as PVA, PAA). Furthermore, the immune suppressive drug can also be directly conjugated to antigen or conjugated to the antigen via a linker or carrier and used in the patch. The carrier can be a polymer. For example, the poly sialic acid-rapamycin in FIG. 8 of U.S. application Ser. No. 15/723,173 can be used to conjugate to the protein's lysine with EDC coupling (e.g. gluten or antibody drug or gliadin or is peanut antigen protein ara h2) and be used in the patch (e.g. 100 ug˜15 mg) instead of the mixture of antigen and drug. The FIG. 12 shows examples of 3 different formats of the antigen-drug conjugate.

When liposome is used, either the drug or both the antigen and immune suppressive drug can be encapsulated in the liposome. Dendritic cell is abundant in skin, adding DC regulating drug with antigen/allergen in a patch can be effective to induce tolerance. Besides being applied topically, the mixture or conjugate can also be injected or taken orally to induce immune tolerance and to treat auto immune disease/allergy.

The topical formulation or implant can contain either antigen+drug or antigen-drug conjugate or encapsulated antigen/drug (e.g. in microsphere or liposome) or their combinations. The antigen can be either in the form of crude antigen (e.g. peanut extract, gluten) or purified antigen (e.g. peanut antigen protein ara h2, gliadin) or antigen-drug conjugate or encapsulated antigen (e.g. in microsphere or liposome) or their mixture.

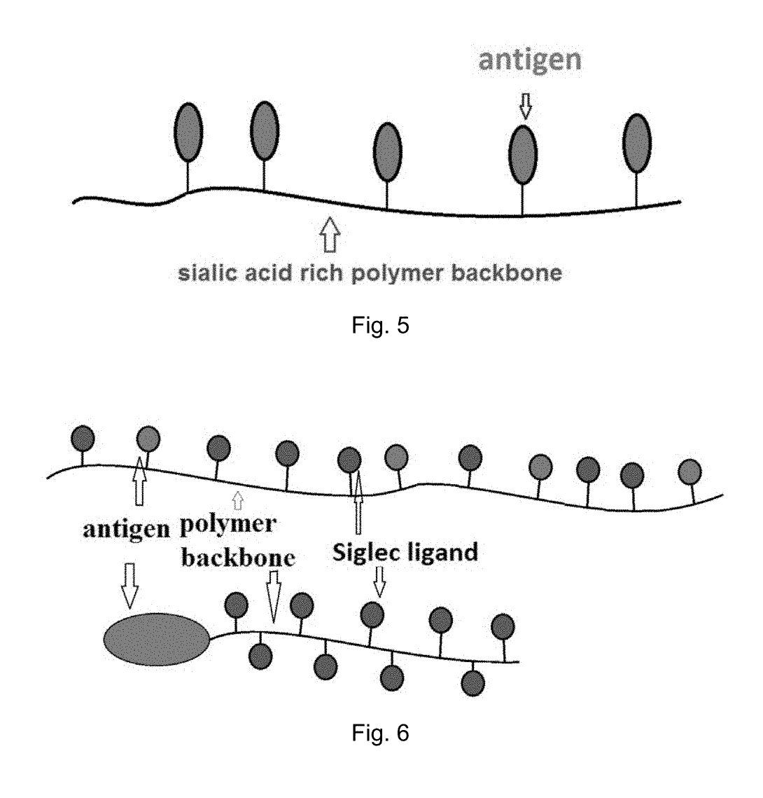

In another format, as shown in FIG. 5, the Epitope(antigen)-Sialic acid rich polymer conjugate or Epitope(antigen)—Siglec ligand rich polymer conjugate can be used to treat autoimmune disease or allergy or to induce immune tolerance, which can be either injected or implanted (being encapsulated inside the implant) or applied topically. The antigen/epitope can be either B cell antigen or T cell antigen or their combination. For example, the lysine group of the antigen can be used to conjugate to the —COOH group of the sialic acid with well known EDC coupling method. The pharmaceutically acceptable amount of conjugate can also be used, as long as it can produce satisfactory therapeutic (e.g. immune tolerance) effect, which can be determined experimentally by screening and testing with well-known protocol.

The term Sialic acid rich polymer means a polymer having multiple sialic acids or siglec ligand conjugated to its back bone. The back bone can be a branched or linear polymer or dendrimer such as synthetic polymer PVA, PAA, polyamine, or nature polymer such as polysialic acid, carbohydrate. The sialic acid or sialic acid containing fragments or siglec ligands are conjugated to the polymer back bone. Sialic acid polymer contains either α2,3 or α2,6 or α2,8 sialoside or sialic acid or their derivatives (e.g. those described in J Immunol. 2006 Sep. 1; 177(5):2994-3003, US patent application U.S. Pat. No. 9,522,183 and U.S. Pat. No. 8,357,671) that can bind with Siglec. The oligo/poly sialic acid with α2,8 linkage backbone itself is also a sialic acid rich polymer. The sialic acid rich polymer can also contains the mixture of different sialoside, sialic acid and/or their derivatives on its backbone. The liposome having sialic acid or sialoside attached on its surface can also be regarded as a sialic acid rich polymer (e.g. those described in U.S. Pat. No. 9,522,183).

There are many sialic acid/siglec ligand rich polymer suitable for the current application can be readily found in the literature, for example, those described in J Immunol. 2006 Sep. 1; 177(5):2994-3003, Nat Chem Biol. 2014 January; 10(1):69-75, J Am Chem Soc. 2013 Dec. 11; 135(49):18280-18283, J Immunol. 2014 Nov. 1; 193(9):4312-21, J Allergy Clin Immunol. 2017 January; 139(1):366-369.e2, Angew Chem Int Ed Engl. 2015 Dec. 21; 54(52):15782-8, Proc Natl Acad Sci USA. 2009 Feb. 24; 106(8):2500-5, J Exp Med. 2010 Jan. 18; 207(1):173-87, J Immunol. 2013 Aug. 15; 191(4):1724-31, Proc Natl Acad Sci USA. 2016 Sep. 13; 113(37):10304-9, J Clin Invest. 2013 July; 123(7):3074-83, Proc Natl Acad Sci USA. 2016 Mar. 22; 113(12):3329-34, U.S. Pat. No. 9,180,182 and U.S. Pat. No. 9,552,183. These sialic acid/siglec ligand rich polymers can be readily adopted for the current inventions. In some embodiments each polymer is conjugated with more than 10 copies of antigen.

Using epitope (antigen)-sialic acid rich polymer conjugate, the antigen will bind with the auto immune T cell or B cell clones, which will guide the conjugated sialic acid rich polymer to inactivate these antigen specific auto immune T cell or B cell clones selectively.

FIG. 6 shows examples of the conjugate containing sialic acid/siglec ligand suitable for the current inventions. Optional linkers can be added between the antigen and the polymer and/or between siglec ligand and the polymer.

When liposome expressing both antigen and siglec ligand is used (e.g. those described in the current invention and those in J Clin Invest. 2013 July; 123(7):3074-83, J Immunol. 2013 Aug. 15; 191(4): 1724-31 and U.S. Pat. No. 9,552,183), the liposome can further encapsulate immuno suppressive drug such as rapamycin. For example, each liposome particle can contain pharmaceutical effective amount of rapamycin (e.g. 1%˜50% liposome weight of rapamycin). This will further increase the efficacy to induce immuno tolerance and treating auto immune diseases/allergy.

Another format suitable for the current application is to use microsphere. The term microsphere include particles from nano meter size to micrometers (e.g. 50 nm˜50 um in diameter). Preferably the microsphere is bio degradable (e.g. made of biodegradable polymer such as poly(lactidecoglycolide)(PLGA)), the microsphere can further encapsulate immune suppressive drug such as rapamycin (e.g. 1%˜80% weight of the microsphere).

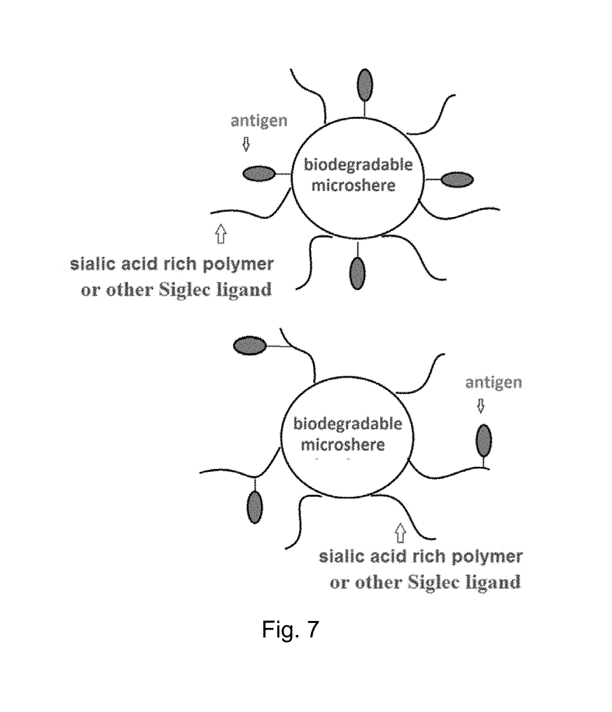

FIG. 7 shows schematic examples of the structure of the microsphere based agent to induce immune tolerance and treating auto immune diseases/allergy. For example, the microsphere can be biodegradable synthetic polymer such as PLGA. Immune suppressive drug such as rapamycin (e.g. 1%˜80% weight of the microsphere) is encapsulated. The size of the microsphere is 3 um or 300 nm. Sialic acid rich polymer or other siglec ligand is conjugated to the surface of the microsphere directly or with a linker, antigen is also conjugated to the surface of the microsphere directly or with a linker. Alternatively, the Sialic acid rich polymer is conjugated to the surface of the microsphere directly or with a linker and the antigen is conjugated to the Sialic acid rich polymer. The antigen can also be encapsulated in the microsphere as well. Alternatively, the drug (immunosuppressant) can be conjugated to the surface of the microsphere or conjugated to the sialic acid rich polymer instead of being encapsulated. Examples of microsphere suitable for the current application can be readily adopted from the disclosure in the publications such as those in patent application U.S. Ser. No. 13/880,778, U.S. Ser. No. 14/934,135, CA 2910579, U.S. Ser. No. 13/084,662 and U.S. Pat. No. 8,652,487 and other patent application filed by Selecta Biosciences. It can be used to treat autoimmune disease or allergy or to induce immune tolerance, which can be either injected or implanted (being encapsulated inside the implant) or applied topically to the patient. The pharmaceutically acceptable amount of these types of conjugate can also be used, as long as it can produce satisfactory therapeutical (e.g. immune tolerance) effect, which can be determined experimentally by screening and testing with well-known protocol.

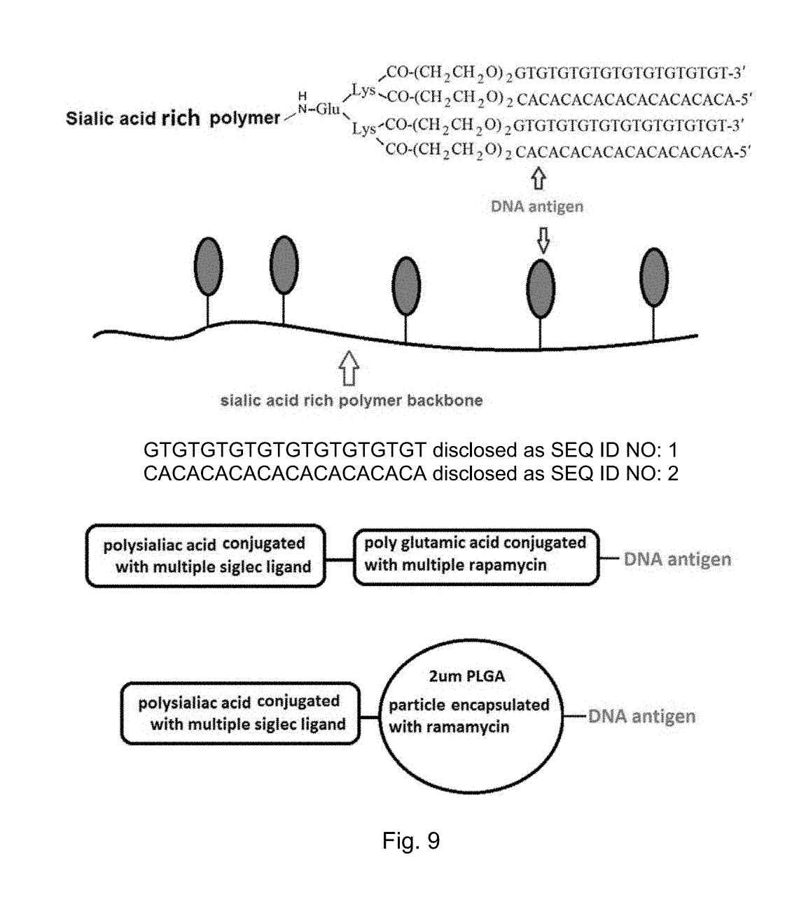

Another format suitable for the current application is to use polymer carrier conjugated with antigen, siglec ligand and/or other immunosuppressant, which is shown in the FIG. 8. Alternatively, both siglec ligand and other immunosuppressant can be conjugated to the antigen. The FIG. 9 shows different formats suitable for the current invention. The polymer conjugated with multiple antigen (e.g. 1-100), multiple siglec ligands (e.g. 5˜500 copies) and multiple copies of other immunosuppressant is essentially the previous described polymer conjugated with antigen and siglec ligand, which is further conjugated with multiple immunosuppressant molecules (e.g. 5˜500 molecules). Alternatively the polymer conjugated with multiple immunosuppressant molecules and multiple siglec ligands can be conjugated to one antigen molecule. Alternatively, multiple immunosuppressant molecules and multiple siglec ligands can be conjugated to one antigen molecule directly or with linker but without polymer carrier. Alternatively, one or more polymer conjugated with multiple immunosuppressant molecules and one or more multiple polymer conjugated with siglec ligands can be conjugated to one antigen molecule. Alternatively, one or more polymer conjugated with multiple immunosuppressant molecules and one or more multiple polymer conjugated with siglec ligands can be conjugated together and then conjugated to one antigen molecule. Other molecule that can promote TB reg expansion (e.g. IL-2 and/or TGF-β and/or PD-L1) can also be conjugated.

They can be used to treat autoimmune disease or allergy or to induce immune tolerance caused by the antigen used to construct these conjugate, which can be either injected or implanted (being encapsulated inside the implant) or applied topically to the subject in need. The pharmaceutically acceptable amount of conjugate in pharmaceutically acceptable formulation can be used, as long as it can produce satisfactory therapeutical (e.g. immune tolerance) effect, which can be determined experimentally by screening and testing with well-known protocol. This method can be used to treat antigen specific autoimmune disease or allergy.

Examples of Sialic acid rich polymer-Antigen conjugate for systemic lupus erythematosus are shown in the FIG. 9. The sialic acid polymer-Antigen conjugate for SLE treatment has the structure of DNA-linker-Sialic acid polymer. In one example, the patient having SLE will receive 200 mg˜1 g of the said conjugate as weekly i.v. injection to treat SLE.

The transdermal delivery system using the combination of antigen and immune suppressant agent are used for allergy, autoimmune diseases and antidrug antibody treatment. When the immune suppressant agent in the above example and methods is replaced with immune enhancing agent (e.g. vaccine adjuvant such as TLR agonist) and the antigen is a pathogen antigen, the transdermal delivery system becomes a vaccine or booster for the pathogen antigen. For example, the transdermal delivery system is a skin patch containing co-formulated immune enhancing agent together with pathogen antigen with optional transdermal delivery enhancer (e.g. azone, fatty acid, hyaluronic acid) in Viaskin® patch or similar dermal patch. It can also be a lotion, gel, liquid, spray, film or other dosage form suitable for topically applied to the skin or membrane. Vaccine adjuvant type molecule such as TLR agonists can be used in the current invention such as MPLA, CpG ODNs, imiquimod, poly IC, resiquimod, gardiquimod, R848 and 3M-052. Examples of the antigen can be either synthetic or purified or the mixture made of pathogen. For example, it can be HIV gp-120, it can be flu neuraminidase, it can be the flu virus lysate, it can be HBV surface antigen and it can be tumor cell lysate. Using these antigens will generate immune response against the pathogen as a vaccine or booster.

In some embodiments, the topical formulations contain 0.1˜100 mg antigen, 0.1˜50 mg TLR agonist in each patch or each mL of gel/lotion/liquid. Transdermal enhancing agent can be added to it as well such as DMSO, azone (e.g. 1%˜10%), surfactant, fatty acid (e.g. 1%˜10% oleic acid). In one example, the formulations contain 10 mg/mL Flu virus lysate, 5 mg/mL imiquimod, 20 mg/mL SDS in 1×PBS and 5% sucrose and then being lyophilized. The lyophilized powder can be used to prepare a skin patch and attached to the skin at 10˜500 mg powder/patch. In another example, 10˜100 mg HBV surface antigen, 5-50 mg of imiquimod is mixed together and added to a VIASKIN® like dermal patch. It can be applied to the skin twice every week for 2 weeks, each time for 2 day as a vaccine and then applied for 2 days as a booster after 1 month and 3 month to generate immunity against HBV. In another example, 100 mg pathogen antigen, 20 mg of poly IC, 20 mg of imiquimod and 100 mg of DMSO is mixed together and added within a skin patch. It can be applied to the skin twice every week for 2 weeks, each time for 2 day as a vaccine and then applied for 2 days as a booster after 1 month and 3 month to generate immunity against said pathogen. The pathogen antigen can be the antigen peptide that can bind with MHC to form MHC-peptide complex. Using antigen peptide instead of MHC-peptide complex improves transdermal delivery.

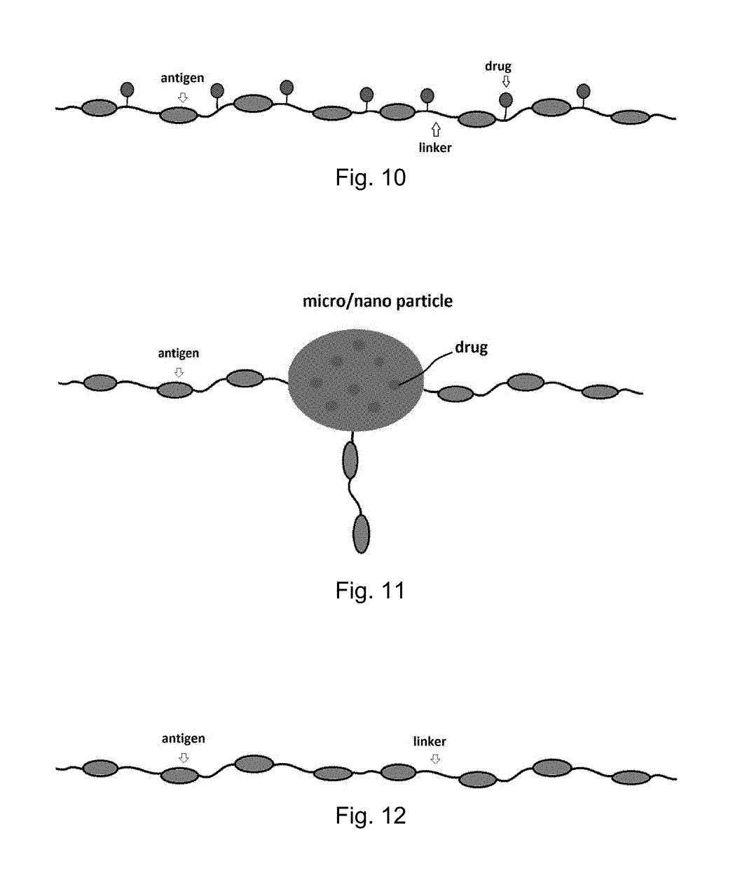

Another format is to connect multiple antigen/epitope with linkers to form a linear polymer and the drug (such as sialic acid or other immunosuppressant listed in the current invention including PD-L1) is conjugated to the linker region or antigen/epitope region or both as shown in FIG. 10. The linker can be either a synthetic polymer such as a PEG (e.g. MW 500 D˜5 KD) or a flexible peptide linker consist of hydrophilic amino acid such as -GGEGGGEGEEEGGGEGGEGGEEGGGEEDGG- (SEQ ID NO: 3). Example of suitable linker can be found in U.S. patent application Ser. Nos. 15/373,483; 15/169,640 and 62/517,994 by the current inventor. XTEN polypeptide from Amunix Inc. can also be used as a peptide linker. When peptide linker is used, the linear polymer can be expressed by recombinant technology if the antigen/epitope is also a peptide or protein that can be linked at its N and C terminal with linker. The drug can be conjugated to the linear polymer directly or with a second linker. The drug conjugated can be either as a single molecule form or multiple molecules form such as in a carrier or encapsulated in nano/micro particle form or in liposome form. In some embodiments, one or more PD-L1 is fused or conjugated with multiple antigen and linkers to form a fusion protein, which can be constructed by expression. Inhibitory ligand that can bind with inhibitory checkpoint receptor (e.g. A2AR, BTLA, CTLA-4, KIR, LAG3, TIM-3, VISTA, CD47 and etc) such as B7-H3, B7-H4 can also be used instead of PD-L1. In some embodiments, the number of antigen/epitope in each polymer backbone is more than 6, preferably more than 8. In some embodiments, the number of antigen/epitope conjugated to each polymer backbone is more than 10. In some embodiments, the number of drug conjugated to each polymer backbone is more than 4. In some embodiments, the number of drug conjugated to each polymer backbone is more than 8. The antigen can be either B cell antigen or T cell antigen in MHC-peptide complex form or the antigen peptide (or its derivative) that can bind with MHC or their combination.

Alternatively, one or more antigen/epitope containing polymer, which each contains one or more antigen/epitope, can be conjugated or coated to a nano/micro particle, which is encapsulated with immune suppressant drug and optionally antigen/epitope. Exemplary scheme can be seen in FIG. 11.

In some embodiments, the drug is not necessary. One format is to connect multiple antigen/epitope with linkers to form a linear polymer. The linker can be either a synthetic polymer such as a PEG (e.g. MW 500 D˜5 KD) or a flexible peptide linker consist of hydrophilic amino acid such as -GGEGGGEGEEEGGGEGGEGGEEGGGEEDGG- (SEQ ID NO: 3). Example of suitable linker can be found in U.S. patent application Ser. Nos. 15/373,483; 15/169,640 and 62/517,994 by the current inventor. XTEN polypeptide from Amunix Inc. can also be used as a peptide linker. When peptide linker is used, the linear polymer can be expressed by recombinant technology if the antigen/epitope is also a peptide or protein that can be linked at its N and C terminal with linker. Exemplary scheme can be seen in FIG. 12.

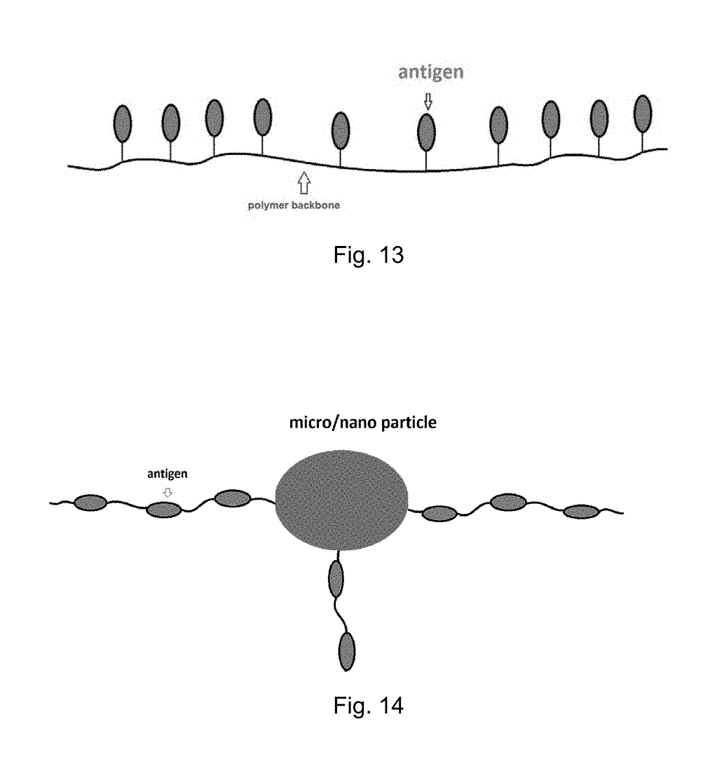

Another format is shown in FIG. 13, which is essentially multiple antigen/epitope conjugated to a polymer back bone (polymer carrier). The polymer back bone can be polypeptide such as Xten from Amunix, synthetic polymer such as poly acrylic acid, carbohydrate includes sialic acid containing polymer, hyaluronic acid, chondroitin sulfate, dextran, carboxyl dextran, cellulose, carboxyl cellulose and their derivatives. The polymer backbone used in previous described prodrug or in previous drug/antigen conjugate can be readily adopted. For example, the average MW of the carbohydrate or other polymer carrier is between 5K˜1000K. In some embodiments, the number of antigen/epitope conjugated to each polymer backbone is more than 8, preferably more than 10. The antigen/epitope can be conjugated to the polymer directly or via a linker. The linker can be either covalent or none-covalent. For example, the linker can be avidin conjugated on polymer bind with the biotin conjugated with antigen/epitope. In some embodiments, the polymer carrier is soluble in aqueous solution.

Similarly, one or more antigen/epitope containing polymer, which each contains one or more antigen/epitope, can be conjugated or coated to a nano/micro particle, which is optionally encapsulated with antigen/epitope. Exemplary scheme can be seen in FIG. 14.

The antigen can be either B cell antigen/epitope or T cell antigen/epitope (e.g. MHC-antigen peptide complex or conjugate; or the peptide antigen that can bind with MHC) or their combination. Examples of them can be found in the current application and related publications and patent applications.

Parvus' NAVACIM® technology use peptide-MHC coated nanoparticles (pMHC-NPs) to delete the high avidity cytotoxic effector T cells, expand a population of autoregulatory memory T cells to target and kill antigen presenting cells (APCs), expand and/or develop populations of Tr1 cells and/or B-regulatory cells in subject to treat corresponding auto immune diseases. It is disclosed in publications and patent applications such as doi: 10.1016/j.immuni.2010.03.015; doi:10.1038/nature16962, doi: 10.1038/nnano.2017.56.; doi: 10.1007/s00109-011-0757-z.; US patent application U.S. Ser. No. 12/044,435, US20090155292A1, US20150125536A1, US20170333540A1, US20170095544A1 and U.S. Pat. No. 8,354,110B2. It has been shown that mono specific pMHC-NP can expand cognate autoregulatory T cells or B cells, suppress the recruitment of noncognate specificities, prevent or treat auto immunity disease.

The antigen/epitope (peptide-MHC complex such as NRP-V7-K^{d} or IGRP₂₀₆₋₂₁₄-K^{d} or both) used in these pMHC-NPs can also be used as antigen/epitope for the current invention to treat corresponding autoimmunity disease such as type 1 diabetes (T1D). Other T1D-relevant pMHC can also be used as antigen/epitope for the current invention to treat type 1 diabetes (T1D). The peptide-MHC complex can be either autoimmune-disease-relevant peptides bound to major histocompatibility complex class II (pMHCII) molecule or autoimmune-disease-relevant peptides bound to major histocompatibility complex class I (pMHCI) molecule or their combinations. Examples of these peptide-MHC complex can be found in the prior arts listed above and can be readily used in the current invention to induce corresponding immune tolerance and to prevent/treat corresponding autoimmune disorder listed in the above cited prior arts.

The above cited prior arts use peptide-MHC-coated nanoparticles with diameter less than 100 nm. Bigger particles including micro particle can also be used to coat with peptide-MHC for the same application, e.g. 200 nm˜200 um in diameter, as long as its surface are conjugated with high density of peptide-MHC complex, to generate pMHC-MPs (peptide-MHC-coated microparticles). In some preferred embodiments, it has a size of 500 nm˜10 um in diameter with >0.5 peptide-MHC molecule/100 nm² surface area. Suitable particles can be made of biodegradable material such as PLGA. Example of biodegradable micro particle suitable for medical application and their surface conjugation protocol are well know to a skilled in the art and can be found easily in the publications.

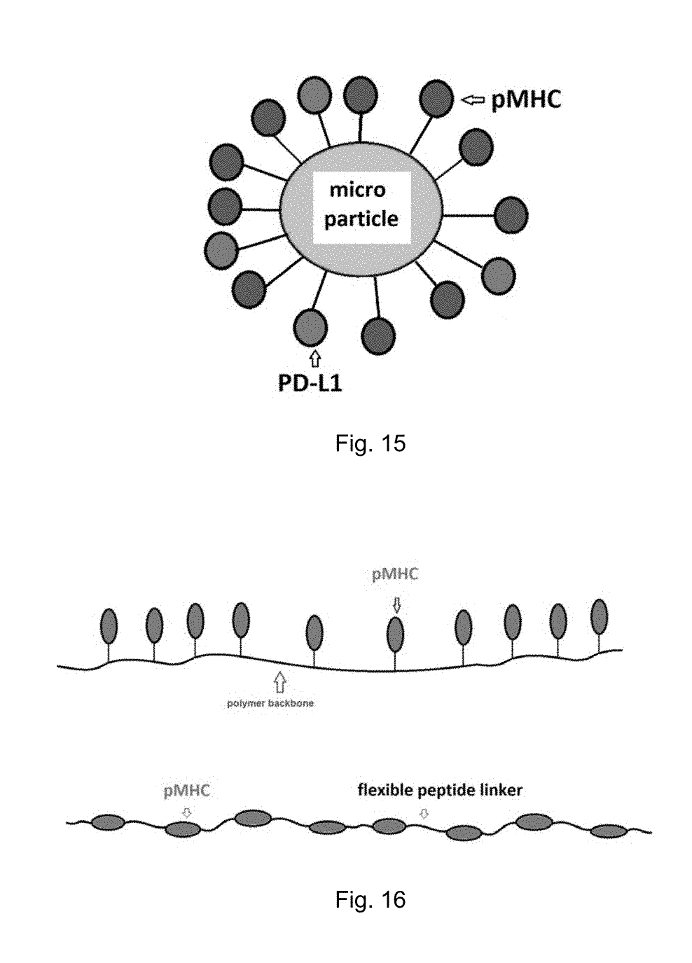

In some embodiments of the current invention, effector molecule such as immunosuppressant drug (e.g. rapamycin or PD-L1) can be further conjugated or encapsulated to the pMHC coated nano/micro particle such as peptide-MHC-coated nanoparticles (pMHC-NPs) cited in the above prior arts (e.g. those used in Parvus' NAVACIM® technology) and those disclosed in the current invention to increase its efficacy. For example, the surface of pMHC-NPs or pMHC-MPs (peptide-MHC-coated microparticles) can be coated with PD-L1 (or its PD-1 binding domain or other PD-1 agonist). Conjugating PD-L1 can effectively inhibit cytotoxic T/B cell and boost Treg/Breg expansion. As shown in FIG. 15, coating additional T/B regulatory cell stimulating molecule/cytokine (e.g. PD-L1, IL-2, TGF-β et.ac.) to pMHC-NP or pMHC-MP is used to increase these T/B regulatory cell expansion and inactivate cytotoxic T/B cell directly. In another example, PD-L2 or other ligand for inhibitory immune check point receptor is coated to the surface of pMHC-NP or pMHC-MP. In another example, immunosuppressant drug such as rapamycin is conjugated to pMHC-NP/pMHC-MP or encapsulated within pMHC-NP/pMHC-MP. In one example, avidin coated NP or MP is prepared according to the protocol in Diabetes 2004 June; 53(6): 1459-1466. https://doi.org/10.2337/diabetes.53.6.1459. Next the mixture solution of biotinylated NRP-V7/H-2K^{d} and biotinylated PD-L1 is added to the avidin coated NP/MP in excess of the binding capacity of the coated avidin (e.g. 2˜5 folds excess) and incubated overnight at 4 C. Next the resulting pMHC-NP/pMHC-MP is washed with PBS 3 times to remove unbound protein. Bigger size NP (e.g. 100˜500 nm) coated with more avidin can also be used instead. Exemplary ratio of V7/H-2K^{d} vs biotinylated PD-L1 used can be between 10:1˜1:3. Other molecule that can promote T/B reg expansion (e.g. T/B reg promoting cytokines such as IL-2 and TGF-β) can also be co-coated to the NP or MP, e.g. by using biotinylated IL-2/TGF-β containing protein mixture described above. Other MHC-peptide complex such as IGRP₂₀₆₋₂₁₄-K^{d} can also be used instead to treat T1D. Other disease related MHC-peptide complex can also be used to treat corresponding disease, for example, pMOG₃₈₋₄₉/IA^{b} (disclosed in doi:10.1038/nature16962) coated NP or MP can also be encapsulated or coated with immunosuppressant to treat experimental autoimmune encephalomyelitis (EAE).

In some embodiments of the current invention, peptide-MHC-coated micro or nanoparticles (pMHC-NP/pMHC-MP) is prepared by coating recombinant single chain MHC complex on the surface of the NP/MP to treat the corresponding autoimmunity diseases instead of the peptide-MHC complex described above. U.S. Ser. No. 08/596,387 disclosed single chain MHC complexes and uses thereof. U.S. Pat. No. 5,869,270 disclosed single chain MHC class II peptide fusion complexes with a presenting peptide covalently linked to the peptide binding grove of the complex. Eur J Immunol. 2000 December; 30(12):3522-32. disclosed recombinant human single-chain MHC-peptide complexes made from E. coli. A skilled in the art can readily adopt the peptide-recombinant single chain MHC complex/conjugate in the prior arts to prepare the peptide-recombinant single chain MHC complex/conjugate coated NP for the current invention. The term MHC complex includes both none-covalent MHC-peptide complex and covalent MHC-peptide conjugate such as those described above. Furthermore mimetic or derivative of MHC-peptide complex can also be used in the current invention to replace the MHC-peptide complex as long as it can bind with the corresponding antigen specific TCR receptor. The MHC-peptide complex mimetic can be readily developed with phage display library or other screening method or computational modeling.

Another format is to use polymer based peptide-MHC oligomer/multimer instead of peptide-MHC coated micro/nanoparticle to induce immune tolerance to the antigen of the MHC-peptide complex and to treat the corresponding auto immune diseases. Preferably the MHC-peptide complex in each polymer is more than 6 copies. In some embodiments the MHC-peptide complex in each polymer is more than 8 copies. In some embodiments the MHC-peptide complex in each polymer is more than 20 copies. The polymer can be a soluble polymer such as the polymer carrier described above. The soluble polymer can be a linear polymer. Examples of MHC multimer can be MHC pentamer, MHC dextramer (e.g. those from www.immudex.com) and those described in US 20100168390 A1 MHC multimers, methods for their generation, labeling and use. The administration protocol can be the same as the pMHC-NPs described above. For example, Immudex dextramer Cat no. WB3329 (peptide: VLFGLGFAI; antigen: IGRP allele: HLA-A*0201) can be used to treat diabetes. In another example, Immudex unlabeled SA-Dextramer Cat no. DX01 is used to mix with biotinylated NRP-V7/H-2K^{d} or the mixture of biotinylated NRP-V7/H-2K^{d} and biotinylated PD-L1 in excess (e.g. 1.2˜2 folds excess of the binding capacity of the streptavidin) and incubated overnight at 4 C. Next the resulting peptide-MHC polymer is dialyzed in PBS to remove unbound peptide-MHC. Other molecule that can promote TB reg expansion (e.g. IL-2 and/or TGF-β) can also be added to bind with SA-Dextramer, e.g. by using biotinylated IL-2/TGF-β containing protein mixture described above. Other MHC-peptide complex such as IGRP₂₀₆₋₂₁₄-K^{d} can also be used instead to treat T1D. The peptide-recombinant single chain MHC complex/conjugate and MHC-peptide complex mimetic can also be used as T cell antigen to build this kind of polymer for the same application.

The above MHC-peptide coated nanoparticle and dextramer based MHC-peptide complex use streptavidin/avidin to conjugate the MHC-peptide complex. Direct conjugation without streptavidin/avidin-biotin binding can also be used instead to incorporate the MHC-peptide complex to the NP/MP or linear polymer using chemical conjugation or other affinity binding such as Fc-protein A interaction. The site-specific conjugation is well known to the skilled in the art and can be adopted from related publications readily. For example the surface mirco/nanoparticle(MP/NP) or polymer can be modified/derivatized to have maleimide groups to allow the —SH (cysteine) of the peptide-MHC to conjugate to them using the well-known maleimide thiol reaction. The protocol for these kind of modification, derivatization and conjugation are well known to the skilled in the arts and can be readily found in the publications and manual of the related reagents. FIG. 16 shows the multiple pMHC is conjugated or expressed in a polymer instead of being coated on particles.

Compounds (e.g. the conjugate, polymer and nano/micro particle disclosed in the current invention) described herein can be administered as a pharmaceutical or medicament formulated with a pharmaceutically acceptable carrier. Accordingly, the compounds may be used in the manufacture of a medicament or pharmaceutical composition. Pharmaceutical compositions of the invention may be formulated as solutions or lyophilized powders for parenteral administration. Powders may be reconstituted by addition of a suitable diluent or other pharmaceutically acceptable carrier prior to use. Liquid formulations may be buffered, isotonic, aqueous solutions. Powders also may be sprayed in dry form. Examples of suitable diluents are normal isotonic saline solution, standard 5% dextrose in water, or buffered sodium or ammonium acetate solution. Such formulations are especially suitable for parenteral administration, but may also be used for oral administration or contained in a metered dose inhaler or nebulizer for insufflation. Compounds may be formulated to include other medically useful drugs or biological agents. The compounds also may be administered in conjunction with the administration of other drugs or biological agents useful for the disease or condition to which the invention compounds are directed. The compound can be formulated in pharmaceutically acceptable carrier. As used herein, the term “pharmaceutically acceptable carrier” refers to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to the desired tissue or a tissue adjacent to the desired tissue. Pharmaceutically acceptable carriers are known to one having ordinary skill in the art may be used, including water or saline. As is known in the art, the components as well as their relative amounts are determined by the intended use and method of delivery. The compositions provided in accordance with the present disclosure are formulated as a solution for delivery into a patient in need thereof, and are, in some embodiments, focused on injection delivery.

Diluent or carriers employed in the compositions can be selected so that they do not diminish the desired effects of the composition. Examples of suitable compositions include aqueous solutions, for example, a saline solution, 5% glucose. Other well-known pharmaceutically acceptable liquid carriers such as alcohols, glycols, esters and amides, may be employed. In certain embodiments, the composition further comprises one or more excipients, such as, but not limited to ionic strength modifying agents, solubility enhancing agents, sugars such as mannitol or sorbitol, pH buffering agent, surfactants, stabilizing polymer, preservatives, and/or co-solvents. In certain embodiments, a polymer matrix or polymeric material is employed as a pharmaceutically acceptable carrier. The polymeric material described herein may comprise natural or unnatural polymers, for example, such as sugars, peptides, protein, laminin, collagen, hyaluronic acid, ionic and non-ionic water soluble polymers; acrylic acid polymers; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; poly(lactic acid), poly(glycolic acid), copolymers of lactic and glycolic acids, or other polymeric agents both natural and synthetic. In certain embodiments, compositions provided herein may be formulated as films, gels, foams, or and other dosage forms. Suitable ionic strength modifying agents include, for example, glycerin, propylene glycol, mannitol, glucose, dextrose, sorbitol, sodium chloride, potassium chloride, and other electrolytes. Suitable pH buffering agents for use in the compositions herein include, for example, acetate, borate, carbonate, citrate, and phosphate buffers, as well as hydrochloric acid, sodium hydroxide, magnesium oxide, monopotassium phosphate, bicarbonate, ammonia, carbonic acid, hydrochloric acid, sodium citrate, citric acid, acetic acid, disodium hydrogen phosphate, borax, boric acid, sodium hydroxide, diethyl barbituric acid, and proteins, as well as various biological buffers, for example, TAPS, Bicine, Tris, Tricine, HEPES, TES, MOPS, PIPES, cacodylate, or IVIES. In certain embodiments, the pH buffer system (e.g., sodium phosphate, sodium acetate, sodium citrate, sodium borate or boric acid) is added to maintain a pH within the range of from about pH 4 to about pH 8, or about pH 5 to about pH 8, or about pH 6 to about pH 8, or about pH 7 to about pH 8.

In some embodiments the said parenteral composition/formulation further include a viscosity enhancing agent to increase its viscosity before or after being injected, which acts as a sustained release formulation. In certain embodiments, the injection has a viscosity greater than 10,000 cps at room temperature. In certain embodiments, the injection has a viscosity greater than 100,000 cps at room temperature. In certain embodiments, the injection has a viscosity greater than 5,000,000 cps at room temperature. In certain embodiments, the injection has a viscosity of 11,000,000 cps at room temperature. Example of the viscosity enhancing agent can be found readily from known pharmaceutical acceptable excipient such as hyaluronic acid, starch and carbomer. In some embodiments, the viscosity enhancing agent is biodegradable. The injection formulation can also be a thermal phase changing formulation. Thermal phase changing formulation is a formulation that change its phase from liquid at low temperature or room temperature (25 C) to semisolid/gel when temperature increases to body temperature (37 C), which can use poloxamer as excipient. A thermal phase changing injectable formulation can be given as either subcutaneous injection or intramuscular injections or intradermal injections to induce antigen specific immune tolerance and treat corresponding auto immune diseases or allergy. It has low viscosity at low or room temperature but high viscosity at body temperature. The preparation of this kind of high viscosity formulation and thermal phase changing injectable formulation can be adopted from related publications readily by the skilled in the art and are described previously in the current invention.

As employed herein, the phrase “an effective amount,” refers to a dose sufficient to provide concentrations high enough to impart a beneficial effect on the recipient thereof. The specific therapeutically effective dose level for any particular subject will depend upon a variety of factors including the disorder being treated, the severity of the disorder, the activity of the specific compound, the route of administration, the rate of clearance of the compound, the duration of treatment, the drugs used in combination or coincident with the compound, the age, body weight, sex, diet, and general health of the subject, and like factors well known in the medical arts and sciences. Various general considerations taken into account in determining the “therapeutically effective amount” are known to those of skill in the art and are described. Dosage levels typically fall in the range of about 0.001 up to 10 mg/kg/day; with levels in the range of about 0.05 up to 5 mg/kg/day are generally applicable. A compound can be administered parenterally, such as intravascularly, intravenously, intraarterially, intramuscularly, subcutaneously, or the like. Administration can also be orally, nasally, rectally, transdermally or inhalationally via an aerosol. The compound may be administered as a bolus, or slowly infused. A therapeutically effective dose can be estimated initially from cell culture assays by determining an IC50. A dose can then be formulated in animal models to achieve a circulating plasma concentration range that includes the IC50 as determined in cell culture. Such information can be used to more accurately determine useful initial doses in humans. Levels of drug in plasma may be measured, for example, by HPLC. The exact formulation, route of administration and dosage can be chosen by the individual physician in view of the patient's condition. In some embodiments, the compound is injected 1 mg/kg˜10 mg/kg to a subject in need either IV or SQ once a week for 2 months. In some embodiments, the compound is injected 1 mg/kg˜10 mg/kg either IV or SQ once per two week for 3 months.

In the current application, the “/” mark means “and” and/or “or” and/or their combination. Unless otherwise defined, all technical and scientific terms used herein have the same meaning as commonly understood by one of ordinary skill in the art to which this invention belongs. All patents and publications mentioned in this specification are indicative of the level of those skilled in the art to which the invention pertains. All patents and publications are herein incorporated by reference to the same extent as if each individual publication was specifically and individually indicated to be incorporated by reference. The inventions described above involve many well-known chemistry, instruments, methods and skills. A skilled person can easily find the knowledge from text books such as the chemistry textbooks, scientific journal papers and other well-known reference sources.

1. A polymer conjugate to induce immune tolerance comprising an antigen causing the immune intolerance and an immunosuppressant conjugated to a linear polymer.

2. The conjugate according to claim 1, wherein the antigen is B cell antigen.

3. The conjugate according to claim 1, wherein the antigen is T cell antigen in MHC-peptide complex form.

4. The conjugate according to claim 1, wherein the immunosuppressant is selected from rapamycin, fujimycin and methotrexate.

5. The conjugate according to claim 1, wherein the first immunosuppressant is PD-L1.

6. A method to induce immune tolerance in a subject, comprising administering to the subject a linear polymer conjugate comprising an antigen causing the immune intolerance and an immunosuppressant.

7. The method according to claim 6, wherein the antigen is B cell antigen.

8. The method according to claim 6, wherein the antigen is T cell antigen in MHC-peptide complex form.

9. The method according to claim 6, wherein the immunosuppressant is selected from rapamycin, fujimycin and PD-L1.

10. The method according to claim 6, wherein the immune intolerance is a condition selected from autoimmune disease, allergy and anti drug antibody.
